(19) United States
(12) Patent Application Publication
(10) Pub. No.: US 2011/0293747 A1
(43) Pub. Date: Dec. 1, 2011

(54) COLON CLEANSING SOLUTION

(75) Inventor: Mark V. CLEVELAND, Duxbury, MA (US)

(73) Assignee: BRAINTREE LABORATORIES, INC., Braintree, MA (US)

(21) Appl. No.: 13/206,948

(22) Filed: Aug. 10, 2011

Related U.S. Application Data
(63) Continuation of application No. 12/109,827, filed on Apr. 25, 2008, now abandoned.
(60) Provisional application No. 61/123,933, filed on Apr. 11, 2008.

(51) Int. Cl. A61K 33/04; A61K 33/14; A61P 1/10; A61K 33/06

(52) U.S. Cl. 424/679; 424/682; 424/709

(57) ABSTRACT

The field of colonic diagnostic and surgical procedures is hampered by the lack of optimal means available to cleanse the colon. A compromise between convenient, distasteful, solid or low volume, hyperosmotic solutions which cause considerable fluid and electrolyte imbalances in patients and large volume, difficult to consume, iso-osmotic solutions has had to be made heretofore. This invention describes a low volume, hyper-osmotic solution consisting of sulfate salts with and with out polyethylene glycol, and further comprising a sweetener and/or flavoring. Unlike prior art, this composition is useful for the cleansing of the bowel and, in lower volumes, as a laxative, without producing clinically significant changes in bodily function.

### BACKGROUND OF THE INVENTION

1. Field of the Invention

The field of this invention is medicine. More specifically, the invention relates to cleansing of the colon in preparation for surgical or diagnostic procedures.

2. Background

In sigmoidoscopy, colonoscopy, radiographic examination, preparation for patients undergoing bowel surgery, and other medical or diagnostic procedures on the colon, it is important that the colon be thoroughly purged and cleaned. In particular, it is essential that as much fecal matter as possible be removed from the colon to permit adequate visualization of the intestinal mucosa. This is important prior to, for example, diagnostic procedures such as flexible sigmoidoscopy or colonoscopy, diagnostic examinations widely performed to screen patients for diseases of the colon. In addition, it is important that the intestines be cleansed thoroughly in order to obtain satisfactory radiographs of the colon. The same condition also applies when the colon is preoperatively prepared for surgery, where removal of fecal waste materials is critically important for patient safety.

Large volume orally administered compositions have been developed for use as gastrointestinal washes for diagnostic purposes or for use as cathartic laxatives. Such orally administered preparations are usually formulated as dilute or isotonic solutions of electrolytes such as sodium sulfate, sodium bicarbonate, sodium chloride and potassium chloride. These orally administered compositions are useful in the rapid cleansing of the colon for diagnostic purposes. These formulations may include other agents such as polyethylene glycol. These formulations have generally been administered in a quantity of about four liters as isotonic solutions. One example composition is GoLYTELY® formulated according to the following: polyethylene glycol 59 g, sodium sulfate 5.68 g, sodium bicarbonate 1.69 g, sodium chloride 1.46 g, potassium chloride 0.745 g and water to make up one liter (Davis et al. Gastroenterology 1980; 78:991-995).

Commercially available products embodying these formulations sometimes utilize polyethylene glycol, a non-absorbable osmotic agent, with an isotonic mixture of electrolytes for replenishment, so that patients do not become dehydrated or experience clinically significant electrolyte shifts. Because the solutions are isotonic, patients are required to ingest a significant amount of volume of these solutions, up to one eight ounce glass every ten minutes for a total of one gallon of fluid, to achieve effective purging.

Sodium sulfate and phosphate salts have been used as laxatives when diluted in about 300 mL of concentrated solution and taken in tablespoon sized (15 ml) daily doses. An example of this use is Glauber's Salt's (containing sodium sulfate). However, because of their small volumes, when used in this fashion they do not sufficiently clean the colon for diagnostic or surgical procedures. Also these small volume preparations do not contain polyethylene glycol. Sodium sulfate combined with polyethylene glycol and various other salts, administered in large volumes (1 gallon) over a short period of time is an effective gastrointestinal lavage, which cleanses the colon prior to colonoscopy or surgical procedures as described above.

However, the large volume required for effective use of this type of formulation for lavage is frequently associated with distention, nausea, vomiting and significant patient discomfort. Thus, while these formulations are generally effective, they are not well tolerated. Another drawback of these prior art preparations is their unpleasant, bitter, saline taste. This can promote nausea and vomiting in sensitive patients, thereby preventing ingestion. It is difficult to overcome this unpleasant taste.

In an attempt to avoid the problems associated with the high volume types of preparations, other investigators have utilized ingestible preparations which consist of aqueous solutions of concentrated phosphate salts. The aqueous phosphate salt concentrate produces a tremendous osmotic effect on the intra-luminal contents of the bowel and therefore, evacuation of the bowel occurs with a large influx of water and electrolytes into the colon from the body. These phosphate salt preparations have been developed for the purpose of decreasing the volume required in colonic purgations. One such preparation basically is comprised of 480 g per liter monobasic sodium phosphate and 180 g per liter dibasic sodium phosphate in stabilized buffered aqueous solution and is sold under the brand name Fleets Phospho-Soda.™ Patients are typically required to take two (2) three ounce doses of this preparation, separated by a three to 12 hour interval for a total of six ounces (180 ml). Additionally, non-aqueous tablet or capsule formulations of sodium phosphates and sulfates have been used (see, e.g., U.S. Pat. Nos. 5,997,906, 6,162,464, and 5,616,346).

However, these small volume sulfate/phosphate solutions and non-aqueous formulations have been shown to cause massive electrolyte and fluid shifts that are clinically significant to the patient (US Food and Drug Administration, Center for Drug Evaluation and Research, Sep. 17, 2001; 2002 Physician's Desk Reference, prescribing information for Fleet's Phospho Soda and InKine Pharmaceutical's Visicol®). The terms “clinically significant” as used herein are meant to convey alterations in blood chemistry that are outside the normal upper or lower limits of their normal range or other untoward effects. These solutions are hyperosmotic; that is the electrolyte concentration of the solution is much higher than the electrolyte concentration in the human body. Available products, such as Fleet's Phospho-Soda, and the solid dosage form such as Visicol tablets (sodium phosphate salts) are examples of small volume electrolyte preparations.

To overcome the risks and electrolyte disturbances that occur with the small volume laxative preparations, large volume “lavage” solutions were developed to be isotonic. Preparing a patient for a surgical or diagnostic procedure on the colon with such an isotonic lavage would result in only minimal fluid and electrolyte shifts in the patient. GoLytely®, NuLytely®, and CoLyte® are examples of such large volume lavages. Because these lavages are isotonic, the patient experiences minimal, non-clinically significant fluid and electrolyte shifts, if any, upon their administration.

From the foregoing, it can be seen that the two approaches to colonic lavage have significant drawbacks that have not been resolved by prior attempts. Thus, it is desirable to have a small volume orally administered colonic purgative formulation which may be easily and conveniently administered and which avoids the clinically significant problems and objectionable tastes of known formulations. It can also be seen that it is desirable to have such a purgative formulation which may be administered without the large volumes necessary in conventional formulations and which avoids other potentially irritant chemicals or chemicals which could effect osmolality. In the nearly 20 years since the advent of large volume colonic lavage solutions, there has not been success in discovering an effective small volume gastrointestinal cleansing preparation that minimized fluid or electrolyte shifts. Concentrating the large volume lavages into smaller volumes does not achieve the same effectiveness, and is not as safe. This is because the components are not soluble in the small volumes necessary and because the concentrations are such that dangerous electrolyte shifts could occur.

### SUMMARY OF THE INVENTION

A new improved concentrated colonic purgative formulation made from poorly absorbable inorganic salts with or without polyethylene glycol (PEG), in a small volume of water is described. This purgative formulation induces a colon cleansing catharsis after oral ingestion without clinically significant alteration of sodium, chloride, bicarbonate, potassium, calcium, and phosphate electrolyte level and balance, and/or other untoward effects on the recipient.

The disclosed colonic purgative formulations provide safe and effective purgative activity at lower dosages of sulfate salt than prior art sodium phosphate tablets, and solutions of mixtures of phosphates and sulfates. In addition, a lower volume of fluid is ingested and there are no clinically significant changes in body electrolyte chemistry. This colonic purgative can be administered with a minimum amount of patient discomfort and is better tolerated than prior art purgatives.

In one aspect, a composition for inducing purgation of the colon of a patient is provided that comprises a small volume, i.e. less than about 500 ml, of an aqueous hypertonic solution which comprises an effective amount of Na₂SO₄, an effective amount of MgSO₄, an effective amount of K₂SO₄, and an effective amount of PEG, wherein the composition does not produce any clinically significant electrolyte shifts and does not include phosphate. In some embodiments, the composition comprises between about 2 g and about 40 g of Na₂SO₄, between about 2 g and about 20 g of MgSO₄, between about 1 g and about 10 g of K₂SO₄, and between about 0.1 g and about 50 g of PEG. In some embodiments, the solution is less than 500 ml. In other embodiments, the volume is between about 100 ml and about 500 ml.

In other embodiments, the composition comprises about 20 g of Na₂SO₄, about 20 g of MgSO₄, about 3 g of K₂SO₄, and about 34 g of PEG.

In another aspect, a composition for inducing purgation of the colon of a patient is provided that comprises a small volume, i.e. less than about 500 ml, of an aqueous hypertonic solution consisting essentially of an effective amount of one or more salts selected from the group consisting of Na₂SO₄, MgSO₄, and K₂SO₄, and an effective amount of PEG, wherein the composition does not produce any clinically significant electrolyte shifts and does not include phosphate.

In some embodiments, the solution has a volume of less than 500 ml. In other embodiments, the solution is from about 100 ml to about 500 ml in volume. In particular embodiments, the solution consists essentially of about 20 g of Na₂SO₄, about 20 g of MgSO₄, about 3 g of K₂SO₄, and about 34 g of PEG in about 330 ml of water.

In another aspect, a composition for inducing purgation of the colon of a patient is provided, that comprises a small volume, i.e. less than about 500 ml, of an aqueous hypertonic solution comprising an effective amount of Na₂SO₄, an effective amount of MgSO₄, and an effective amount of K₂SO₄, wherein the composition does not produce any clinically significant electrolyte shifts and does not include phosphate.

In some embodiments, the solution comprises between about 2 g and about 40 g of Na₂SO₄, between about 2 g and about 20 g of MgSO₄, and between about 1 g and about 10 g of K₂SO₄. In other embodiments, the composition for inducing purgation of the colon of a patient comprises a small volume, of an aqueous hypertonic solution consisting essentially of an effective amount of Na₂SO₄, an effective amount of MgSO₄, and an effective amount of K₂SO₄, wherein the composition does not produce any clinically significant electrolyte shifts and does not include phosphate. In certain embodiments, the solution administered consists essentially of about 20 g of Na₂SO₄, about 20 g of MgSO₄, and about 3 g of K₂SO₄.

In another aspect, a method for inducing colonic purgation in a patient is provided. This method comprises the steps of orally administering an effective amount of one of the compositions provided herein to a patient and allowing the administered composition to induce colonic purgation.

In some embodiments, the solution administered to the patient consists essentially of about 20 grams of Na₂SO₄, about 20 grams of MgSO₄, about 3 grams of K₂SO₄, and about 34 grams of PEG in about 330 ml of water. In other embodiments, the effective amount of the composition is administered to the patient in two or more doses within a treatment period. In one embodiment, the composition is administered as two (2) one-half doses separated by a few hours. In some embodiments, about 100 ml to about 500 ml of the solution is administered to the patient. In other embodiments, the solution administered to the patient comprises an effective amount of two or more salts selected from the group consisting of Na₂SO₄, MgSO₄, and K₂SO₄, and an effective amount of PEG. In still further embodiments, the solution administered to the patient contains no PEG.

In another aspect, a composition for inducing purgation of the colon of a patient described above is provided which further comprises a sweetener. This composition has a perceived saltiness equivalent to from about 0.2% to about 2.6% sodium chloride in water, and contains from about 0.01% to about 0.1% of a sweetener. In particular embodiments, the sweetener is selected from the group consisting of a chlorinated sucrose isomer, acesulfame potassium, saccharin, and mixtures thereof.

In some embodiments, the chlorinated sucrose isomer is Sucralose®. In other embodiments, the sweetener is a mixture of chlorinated sucrose isomer and acesulfame potassium in a 1 to from about 4 to about 6 ratio by weight, respectively. In other embodiments, the composition for inducing purgation of the colon of a patient comprises from about 0.04% to about 0.09% of the sweetener and where the perceived saltiness of the bowel cleanser is equivalent to from about 1.8% to about 2.2% sodium chloride in water.

In still other embodiments, the composition for inducing purgation of the colon of a patient comprises from about 0.04% to about 0.06% of the sweetener, where the sweetener is selected from the group consisting of the chlorinated sucrose isomer, acesulfame potassium, saccharin and mixtures thereof. In other embodiments, the composition for inducing purgation of the colon of a patient comprises from about 0.01% to about 0.04% of the sweetener, and where the perceived saltiness of the bowel cleanser is equivalent to from about 0.2% to about 0.6% sodium chloride in water. In further embodiments, the composition for inducing purgation of the colon of a patient comprises from about 0.01% to about 0.03% of the sweetener, where the sweetener is selected from the group consisting of chlorinated sucrose isomer, acesulfame potassium, saccharin, and mixtures thereof. In some embodiments the composition for inducing purgation of the colon of a patient further comprises a flavoring. In particular embodiments, the flavoring is selected from the group consisting of mangosteen, cola, ginger ale, and combinations thereof. In some embodiments the composition includes from about 0.3% to about 2.3% of the flavoring.

In another aspect, a method is provided for reducing the saltiness of an orally consumed substance having a perceived saltiness equivalent to from 0.2 to 2.6% sodium chloride in water. This method comprises combining the orally consumed substance with from 0.01% to 0.1% of a sweetener. In particular embodiments, the sweetener is selected from the group consisting of a chlorinated sucrose isomer, acesulfame potassium, saccharin, and mixtures thereof. In some embodiments, the orally consumed substance comprises a composition for inducing purgation of the colon.

In a still further aspect, a method is provided for improving the palatability of a composition for inducing purgation of the colon of a patient. This method comprises combining a composition for inducing purgation of the colon having a perceived saltiness equivalent to from about 0.2% to about 2.6% sodium chloride in water with from about 0.01% to about 0.07% of a sweetener.

### DETAILED DESCRIPTION

All patents, patent applications and publications cited herein, whether supra or infra, are hereby incorporated by reference in their entireties into this application in order to more fully describe the state of the art as known to those skilled therein as of the date of the invention described and claimed herein.

A safe and effective small volume colonic purgative formulation is described herein that avoids the problems of the prior art, using poorly absorbable sulfate salts optionally with a small quantity of polyethylene glycol.

Hypertonic solutions of non-phosphate salts are effective in producing colonic purgation. Addition of an osmotic laxative agent such as polyethylene glycol may further improve further the results in the improved purgation described herein and reduces the amounts of salts required. Because it is administered in small volumes, these formulations are better tolerated than formulations now used and have a lower risk of adverse reactions.

Mixtures of sulfate salts that omit phosphates (which are avidly absorbed) can be effective to produce colonic purgation. In particular, formulations comprising effective amounts of one or more of the following sulfate salts Na₂SO₄, MgSO₄, and K₂SO₄ are effective. For example, dosage amounts of Na₂SO₄ from about 0.01 g to about 40.0 g can be effective to produce purgation.

As used herein, the term “about” applies to all numeric values, whether or not explicitly indicated. As used herein, the term “about” means a numeric value having a range of ±10% around the recited value. For example, a range of “about 1.5 times to about 2 times” includes the range “1.35 times to 2.2 times” as well as the range “1.65 times to 1.8 times,” and all ranges in between. Doses of from about 0.1 g to about 20.0 g Na₂SO₄, and from about 1.0 g to 10.0 g Na₂SO₄ may be useful. Dosage amounts of MgSO₄ from about 0.01 g to about 40.0 g can be effective to produce purgation. Doses of from about 0.1 g to about 20.0 g Na₂SO₄ may also be advantageously used, as well as dosages of 1.0 to 10.0 g. Dosage amounts of K₂SO₄ from about 0.01 g to about 20.0 g can be effective to produce purgation, and doses of from about 0.1 g to about 10.0 g and from about 0.5 g to about 5.0 g K₂SO₄ may also be useful.

Addition of an osmotic laxative agent, such as polyethylene glycol (PEG) may improve the effectiveness of the above salt mixtures. Doses of PEG from about 1.0 g to about 100 g PEG are effective to produce laxation. Doses from about 10.0 g to about 50 g of PEG are also effective, as is a dose of about 34 g.

For ease of administration, the above mixture of salts can be dissolved in a convenient volume of water. A volume of less than one liter of water is well tolerated by most patients. The mixture can be dissolved in any small volume of water, and volumes of between 100 and 500 ml are useful. The effective dose may be divided and administered to the patient in two or more administrations over an appropriate time period. Generally, two doses administered of equal portions of the effective dose, separated by 6 to 24 hours produce satisfactory purgation.

For many people, the compositions described herein which comprise an effective amount of Na₂SO₄, MgSO₄, and K₂SO₄, have a salty taste. Although the solution does not contain any significant amount of sodium chloride, the compositions have “perceived saltiness.” The term “perceived saltiness” as used herein refers to some solutions of salts (not necessarily NaCl) have a salt taste equivalent to solutions containing from about 0.2% to about 2.6% sodium chloride. This amount of salts with such perceived saltiness can be difficult for many people to ingest without becoming nauseous and even vomiting. Sweeteners and flavorings are used to aid in masking the perceived saltiness of the purgative solutions. Such sweeteners and/or flavorings for use in bowel cleansers usually exclude natural sugars that may be digested in the colon to form hydrogen gas, which may ignite during polypectomy.

While it is possible to add the sweetener and/or flavoring a short time before consumption of the purgative solution and retain at least a portion of the palatability benefits, one goal of the present purgative compositions is to provide stable liquids having increased palatability. Non-limiting examples of sweeteners useful in the formulation of the invention are chlorinated sucrose isomers, such as Sucralose®, Ace-K, and Saccharin and mixtures thereof.

Non-limiting examples of useful flavorings include mangosteen, cola, ginger ale, and combinations thereof.

Reference will now be made to specific examples illustrating the invention. It is to be understood that the examples are provided to illustrate preferred embodiments and that no limitation to the scope of the invention is intended thereby.

### EXAMPLES

### Example 1

### Dietary Preparation and Ingestion of Salt Solution

Each experiment began at midnight on the first day of a two day study period, and was completed at noon on the next day. Subjects were otherwise healthy adults between the ages of 18 and 55. There were no preferences or exclusions based on gender or ethnic background. The subjects did not consume any food or beverages after midnight on day 1. From 6 a.m. until 6 p.m. on day 1 the subjects consumed a clear liquid diet. Clear liquids included strained fruit juices without pulp (apple, white grape, lemonade), water, clear broth or bouillon, coffee or tea (without milk or non-dairy creamer), carbonated and non-carbonated soft drinks, Kool-Aid® (or other fruit flavored drinks), Jell-O® gelatin (without added fruits or toppings), and ice PopSicles° fruit bars. Solid foods, milk, and milk products are not allowed. The subjects kept a record of exactly what they consumed on day 1, and they were asked to consume the same liquids at the same time if and when they did subsequent studies with a different solution.

Subjects reported to the laboratory at 6 p.m. on day 1. At 7 p.m. they ingested the first dose of concentrated salt solution, either Fleet Phospho-Soda or the experimental solution, followed by 8 ounces of water. Eight ounces of water was also ingested at 8, 9, and 10 p.m.

At 5 a.m. on day 2, a second dose of the concentrated salt solution was ingested, followed by 8 ounces of water.

Formulation of Concentrated Salt Solutions:

Fleet Phospho-Soda (C. S. Fleet Co., Inc., Lynchburg, Va. 24506), 90 mL, was added to 240 mL of water, for a volume of 330 mL. One half of this diluted solution was ingested by the subjects on two occasions, at 7 p.m. on day 1 and again at 5 a.m. on day 2. Based on the manufacturer label, the 330 mL of ingested Phospho-Soda solution contained NaH₂PO₄H₂O (43.2 g) and Na₂HPO₄0.7H₂O (16.2 g).

The ingested experimental solutions were also 330 mL in volume, and their composition is shown in the tables below. All salts were obtained from Mallinckrodt (Paris, Ky. 40361) and Polyethylene glycol (PEG) was obtained from J. T. Baker (Phillipsburg, N.J. 08865). One half of each experimental solution was ingested by the subjects on two occasions, at 7 p.m. on day 1 and at 5 a.m. on day 2.

TABLE 1

| | Experimental Solutions (mmoles) | | | | | |
|---|---|---|---|---|---|---|
| Salts | Fleet | A | B | C | D | E |
| NaH₂PO₄•H₂O | 313 | 0 | 0 | 157 | 0 | 0 |
| Na₂HPO₄•7H₂O | 60 | 0 | 0 | 30 | 0 | 0 |
| Na₂SO₄ | 0 | 100 | 125 | 142.5 | 142.5 | 142.5 |
| MgSO₄ | 0 | 100 | 125 | 0 | 142.5 | 142.5 |
| K₂SO₄ | 0 | 0 | 12.5 | 23.75 | 23.75 | 20 |
| KCl | 0 | 5 | 0 | 0 | 0 | |
| KHCO₃ | 0 | 5 | 0 | 0 | 0 | |

TABLE 2

Experimental Solutions (mmoles)

| Salts | Fleet | A | B | C | D | E |
|---|---|---|---|---|---|---|
| Na | 433 | 200 | 250 | 502 | 285 | 285 |
| K | 0 | 10 | 25 | 48 | 48 | 40 |
| Mg | 0 | 200 | 250 | 0 | 285 | 285 |
| SO₄ | 0 | 400 | 525 | 333 | 618 | 610 |
| PO₄ | 11.6 | 0 | 0 | 5.8 | 0 | 0 |
| Cl | 0 | 5 | 0 | 0 | 0 | 0 |
| HCO₃ | 0 | 5 | 0 | 0 | 0 | 0 |

### Observations and Measurements:

Body weight was measured at 6:45 p.m. on day 1, and at noon on day 2. Blood pressure (lying and after standing for 30 seconds) was measured every two hours, starting at 6:45 p.m. on day 1 and finishing at 11:45 a.m. on day 2. Blood was drawn at 6:45 p.m. on day 1 and at 6 a.m., 8 a.m., 10 a.m. and 12 noon on day 2. Blood was analyzed for calcium, sulfate, magnesium, phosphate, sodium, chloride, potassium, bicarbonate, osmolality, albumin, total protein, BUN, creatinine, and hematocrit.

Each stool was quantitatively collected in separate containers and its weight and consistency were measured. The degree to which the stool contained fecal material was graded, using a scale from 0-5 (0 would be similar to urine, 5 would be a large amount of solid fecal material). Stools collected from 7 p.m. (day 1) until 5 a.m. (day 2) were pooled: this pool represents the effects of the first dose of salts. Stools collected from 5 a.m. until 12 noon were pooled; this pool represents the effect of the second dose of salts. The electrolyte composition of the two pooled specimens was measured (osmolality, Na, K, Cl, HCO₃, PO₄, S₄, Ca and Mg). Urine was quantitatively collected from 6 a.m. until 6 p.m. on day 1 (prior to ingestion of salts), from 7 p.m. on day 1 until 5 a.m. on day 2, and from 5 a.m. on day 2 until 12 noon on day 2. Urine was analyzed for sulfate, phosphate, calcium, magnesium and monovalent electrolytes.

### Results

Study results are shown in Tables 3, 4, 5, and 6.

As indicated in Table 3, stool volume averaged 2403 mL in three subjects who ingested the standard dose of Phospho-Soda.

TABLE 3
Fecal and Urine Analysis

| | | | | URINE |
|---|---|---|---|---|
| | FECAL | | | Output |
| | Intake | Output | Change | (mL) |
| Volume (mL) | | | | |
| Phospho-Soda | 1530 | 2403 | −873 | 902 |
| Experimental Solution | | | | |
| A | 1530 | 1510 | 20 | 832 |
| B | 1530 | 2209 | −679 | 789 |
| C | 1530 | 1868 | −338 | 779 |
| D | 1530 | 2202 | −672 | 639 |
| E | 1530 | 2729 | −1199 | 780 |
| Sodium (mEq) | | | | |
| Phospho-Soda | 437 | 397 | 40 | −80 |
| Experimental Solution | | | | |
| A | 200 | 198 | 2 | 89 |
| B | 200 | 302 | −102 | 109 |
| C | 502 | 360 | 142 | 169 |
| D | 285 | 331 | −46 | 132 |
| E | 285 | 369 | −84 | 95 |
| Potassium (mEq) | | | | |
| Phospho-Soda | 0 | 54 | −54 | 29 |
| Experimental Solution | | | | |
| A | 10 | 30 | −20 | 19 |
| B | 20 | 41 | −21 | 21 |
| C | 48 | 34 | 14 | 44 |
| D | 48 | 44 | 4 | 28 |
| E | 40 | 42 | −2 | 24 |
| Chloride (mEq)) | | | | |
| Phospho-Soda | 0 | 41 | −41 | 42 |
| Experimental Solution | | | | |
| A | 5 | 36 | −31 | 53 |
| B | 0 | 71 | −71 | 82 |
| C | 0 | 21 | −21 | 81 |
| D | 0 | 71 | −71 | 86 |
| E | 0 | 81 | −81 | 62 |
| Bicarbonate (mEq) | | | | |
| Phospho-Soda | 0 | 19 | −19 | |
| Experimental Solution | | | | |
| A | 5 | 38 | −33 | 0 |
| B | 0 | 61 | −61 | 0 |
| C | 0 | 16 | −16 | 0 |
| D | 0 | 89 | −89 | 0 |
| E | 0 | 72 | −72 | 0.9 |
| Phosphorous (g) | | | | |
| Phospho-Soda | 10.6 | 6.5 | 4.1 | 1.7 |
| Experimental Solution | | | | |
| A | 0 | 0.1 | −0.1 | 0.3 |
| B | 0 | 0.2 | −0.2 | 0.2 |
| C | 5.8 | 2.3 | 3.5 | 0.3 |
| D | 0 | ND | 0 | 0.4 |
| E | 0 | 0.13 | −0.1 | 0.3 |
| Calcium (mEq) | | | | |
| Phospho-Soda | 0 | 5 | −5 | 1.7 |
| Experimental Solution | | | | |
| A | 0 | 9 | −9 | 7 |
| B | 0 | 11 | −11 | 5 |
| C | 0 | 3 | −3 | 3 |
| D | 0 | 8 | −8 | 8 |
| E | 0 | 17 | −17 | 6 |
| Magnesium (mEq) | | | | |
| Phospho-Soda | 0 | 9 | −9 | 1.8 |
| Experimental Solution | | | | |
| A | 200 | 156 | 44 | 6 |
| B | 200 | 193 | 7 | 5 |
| C | 0 | 3 | −3 | 2 |
| D | 285 | 187 | 98 | 7 |
| E | 285 | 239 | 46 | 7 |
| Sulfate (mEq) | | | | |
| Phospho-Soda | 0 | 12 | −12 | 11 |
| Experimental Solution | | | | |
| A | 400 | 285 | 115 | 65 |
| B | 420 | 370 | 50 | 55 |
| C | 333 | 210 | 123 | 74 |
| D | 618 | 433 | 185 | 63 |
| E | 610 | 478 | 132 | 58 |
| PEG (g) | | | | |
| Phospho-Soda | 0 | 0 | 0 | 0 |
| Experimental Solution | 0 | | | |
| A | 0 | 0 | | |
| B | 0 | 0 | | 0 |
| C | 0 | 0 | | 0 |
| D | 0 | 0 | | 0 |
| E | 34 | 29.1 | | 4.9 |

Table 4 shows that this result was associated with a clinically significant rise in serum phosphate, a clinically significant fall in serum calcium, a clinically significant rise in serum calcium×phosphate product (Ca×P), and a large net gastrointestinal potassium loss of 54 mEq. Serum potassium also fell, but generally stayed in the normal range. However, all subjects had a net negative balance in potassium. Serum phosphorus increased markedly, well outside of the normal range.

TABLE 4
Serum Electrolyte and Mineral Data

| | 645 PM | 600 AM | 800 AM | 10 AM | 1200 PM |
|---|---|---|---|---|---|
| Sodium (mEq/L) | | | | | |
| Phospho-Soda | 138 | 141 | 142 | 143 | 143 |
| Experimental Solution | | | | | |
| A | 138 | 139 | 140 | ND | ND |
| B | 140 | 142 | 141 | 142 | 142 |
| C | 141 | 142 | 144 | 144 | 144 |
| D | 136 | 139 | 138 | 138 | 138 |
| E | 140 | 141 | 142 | 141 | 142 |
| Potassium (mEq/L) | | | | | |
| Phospho-Soda | 4.9 | 3.7 | 3.9 | 4.0 | 3.9 |
| Experimental Solution | | | | | |
| A | 5.4 | 4.0 | 4.2 | ND | ND |
| B | 5.7 | 4.4 | 4.7 | 4.9 | 4.4 |
| C | 5.5 | 4.2 | 4.6 | 4.6 | 4.5 |
| D | 7.3 | 4.2 | 4.6 | 4.2 | 4.2 |
| E | 4.6 | 4.0 | 4.3 | 4.4 | 4.3 |
| Chloride (mEq/L)) | | | | | |
| Phospho-Soda | 103 | 105 | 107 | 107 | 107 |
| Experimental Solution | | | | | |
| A | 107 | 104 | 106 | ND | ND |
| B | 107 | 106 | 108 | 108 | 107 |
| C | 106 | 107 | 109 | 110 | 109 |
| D | 108 | 106 | 107 | 107 | 106 |
| E | 105 | 105 | 107 | 107 | 107 |
| Bicarbonate (mEq/L) | | | | | |
| Phospho-Soda | 23 | 23 | 21 | 22 | 23 |
| Experimental Solution | | | | | |
| A | 21 | 23 | 23 | ND | ND |
| B | 20 | 21 | 19 | 21 | 20 |
| C | 23 | 22 | 22 | 22 | 23 |
| D | 24 | 23 | 21 | 21 | 21 |
| E | 23 | 24 | 23 | 22 | 23 |
| Sulfate (mEq/L) | | | | | |
| Phospho-Soda | 1.63 | 1.68 | 1.52 | 1.75 | 1.70 |
| Experimental Solution | | | | | |
| A | 1.16 | 1.79 | 1.84 | ND | ND |
| B | 1.92 | 1.75 | 1.83 | 1.58 | 1.83 |
| C | 1.38 | 1.86 | 1.54 | 1.70 | 1.78 |
| D | 0.88 | 1.30 | 1.62 | 1.46 | 1.30 |
| E | 1.36 | 1.85 | 2.01 | 1.87 | 1.62 |
| Phosphorous (mg/dL) | | | | | |
| Phospho-Soda | 3.3 | 6.5 | 7.9 | 6.3 | 5.4 |
| Experimental Solution | | | | | |
| A | 2.6 | 3.1 | 2.8 | ND | ND |
| B | 2.8 | 3.1 | 2.8 | 2.8 | 2.9 |
| C | 3.1 | 5.9 | 6.6 | 5.8 | 4.4 |
| D | 3.2 | 2.7 | 2.7 | 2.7 | 2.8 |
| E | 3.3 | 3.3 | 3.3 | 3.2 | 3.2 |
| Calcium (mg/dL) | | | | | |
| Phospho-Soda | 9.2 | 9.1 | 8.9 | 9.0 | 9.1 |
| Experimental Solution | | | | | |
| A | 9.2 | 9.3 | 9.5 | ND | ND |
| B | 9.4 | 9.6 | 9.4 | 9.5 | 9.5 |
| C | 9.4 | 9.3 | 9.3 | 9.2 | 9.5 |
| D | 8.9 | 9.1 | 8.8 | 9.0 | 8.7 |
| E | 9.3 | 9.5 | 9.7 | 9.6 | 9.6 |
| Ca × P | | | | | |
| Phospho-Soda | 30.2 | 59.7 | 70.7 | 56.5 | 48.9 |
| Experimental Solution | | | | | |
| A | 23.9 | 28.8 | 26.6 | ND | ND |
| B | 26.3 | 29.8 | 26.3 | 26.6 | 27.6 |
| C | 29.1 | 54.9 | 61.4 | 53.4 | 41.8 |
| D | 28.5 | 24.6 | 23.8 | 24.3 | 24.4 |
| E | 30.9 | 31.5 | 32.2 | 30.4 | 30.3 |
| Magnesium (mg/dL) | | | | | |
| Phospho-Soda | 2.0 | 2.1 | 2.1 | 2.2 | 2.2 |
| Experimental Solution | | | | | |
| A | 2.3 | 2.6 | 2.6 | ND | ND |
| B | 2.3 | 2.7 | 2.6 | 2.7 | 2.7 |
| C | 2.3 | 2.4 | 2.3 | 2.3 | 2.4 |
| D | 1.8 | 2.0 | 1.9 | 1.9 | 1.9 |
| E | 2.0 | 2.3 | 2.4 | 2.5 | 2.4 |
| Hematocrit | | | | | |
| Phospho-Soda | 40.0 | 42.3 | 41.8 | 43.8 | 43.1 |
| Experimental Solution | | | | | |
| A | 38.5 | 39.8 | 39.3 | ND | ND |
| B | 37.8 | 41.1 | 39.8 | 39.5 | 39.5 |
| C | 35.3 | 36.8 | 37.0 | 36.7 | 37.2 |
| D | 37.1 | 39.7 | 40.1 | 40.2 | 40.8 |
| E | 38.8 | 40.8 | 41.7 | 42.8 | 42.9 |

Solution A contained 100 mmoles of Na₂SO₄ and 100 mmoles of MgSO₄, as well as small amounts of KCl and KHCO₃ to replace anticipated K, Cl, and HCO₃ losses. After ingestion of solution A, stool output (1500) was short of the Phospho-Soda output benchmark (2403 ml) as seen in Table 3.

For solution B K₂SO₄ was substituted for KCl and KHCO₃; the Na₂SO₄ and MgSO₄ contents were each increased to 125 mmoles. Fecal output rose with solution B to 2209 mL, but as shown in Table 4 the potassium losses were unacceptably high.

The effect of adding phosphate salts was investigated using solution C which contained one half of the amount of phosphate in the Fleet Phospho-Soda protocol, and 142.5 mmoles of Na₂SO₄. As seen in Table 3, this solution resulted in 1868 mL of fecal output. However, there was substantial net sodium absorption from this solution, and the serum Ca×P product increased dramatically due to absorbed phosphate. From these results it was concluded that phosphate should be excluded completely from further experimental solutions.

Solution D contained 142.5 mmoles of both Na₂SO₄ and MgSO₄, and 23.75 mmoles of K₂SO₄. This solution resulted in a stool volume of 2202 mL, which was slightly (180 mL) short of benchmark. Electrolyte changes were clinically insignificant with this formulation. Further increase in the ingested amounts of salts would likely be effective however would result in palatability problems with patients.

For solution E, PEG 3350 was added and the K₂SO₄ content reduced slightly as compared to solution D. In two subjects, solution E produced an average fecal output that exceeded the Phospho-Soda benchmark, and the taste was acceptable. This solution caused no increase in Ca×P product, and its effect on potassium balance appeared to be close to zero. A small clinically insignificant change was seen for magnesium, which stayed within the normal range of 1.4 mg/dL to 3.1 mg/dL. Changes in sodium, chloride, sulfate and bicarbonate balance with this solution were considered to be of no clinical significance.

There are two ways to estimate the degree to which the poorly absorbable solutes were absorbed by the intestine. The first involves subtraction of fecal output from oral intake. This method assumes that anything not excreted in the stool by the end of the experiment was absorbed. Using this method, the absorption of phosphate after ingesting of Fleet Phospho-soda was 4.0 g, or 38% of the ingested phosphate load.

The absorption of sulfate after ingestion of solution E was 165 mEq, or 27% of the ingested load. However, the serum sulfate concentration remained well below the level at which calcium sulfate precipitates form, therefore calcium levels remained unchanged. The absorption of magnesium after ingestion of solution E was 66 mEq, or 23% of the ingested load. The second method that can be used involves changes in urine output of the solutes. When a phosphate-free solution was ingested (solution E), urine phosphate excretion was 0.4 g, whereas when 10.6 g of phosphate were ingested (Fleet Phospho-Soda), urine phosphate excretion was 2.1 g (=1.7 g); thus, 16% of the ingested phosphate appeared in the collected urine. By a similar calculation, 10% of ingested sulfate and 2% of ingested magnesium appeared in the collected urine. Intestinal absorption of the ingested electrolytes occurred in the following order of magnitude: P>SO₄>Mg.

The highest observed Ca×P product varied from 62 to 76 with Phospho-Soda which is well in excess of the level at which calcium-phosphate precipitates from. For solution E, Ca×P was from 30 to 37. The Phospho-Soda preparation caused a net gastrointestinal loss of 54 mEq of potassium, whereas solutions D and E caused essentially no loss or gain of potassium.

The serum phosphate concentration increased more than 2-fold after ingestion of Phospho-Soda, whereas the serum sulfate concentration rose only slightly after ingestion of solution E. There were no significant changes in serum magnesium concentration.

Solution E contains three sulfate salts (Na₂SO₄, K₂SO₄ and MgSO₄) as well as PEG. Sulfate, magnesium and polyethylene glycol are poorly absorbed, and ingestion of this solution therefore induces osmotic diarrhea. The sodium content of solution E is less than the sodium content of Phospho-Soda, and solution E contains potassium whereas Phospho-Soda does not. In contrast to Phospho-Soda, solution E does not cause serum phosphate concentration to rise, and does not cause a net gastrointestinal loss of potassium.

Both solutions were associated with approximately 2.5 kg loss in body weight which can be explained by higher water output (in both stool and in urine) than water intake by mouth. To prevent this weight loss, the subjects could ingest an additional 2.5 kg of water, which would increase total water intake to approximately 4 liters. There were no changes in the vital signs of the test subjects, indicating that the observed body water losses caused by ingestion of the two solutions are well tolerated by normal people.

### Example 2

A non-limiting example of a formulation of the present invention is listed in Table 5.

TABLE 5
Formulation Composition

| | Total | Composition | |
|---|---|---|---|
| Ingredient | Dose (g) | (solids %) | Function |
| Na₂SO₄ | 35.02 | 70.72 | Active |
| MgSO₄ | 3.2 | 6.46 | Active |
| K₂SO₄ | 6.26 | 12.64 | Active |
| Sodium Benzoate | 0.136 | 0.27 | Preservative |
| Flavoring agents | 2.7 | 5.45 | Flavoring |
| Sucralose, NF | 2.2 | 4.44 | Flavoring |

Ranges of salts, flavoring agents, and sweeteners useful in the formulations of the present invention are listed in Table 6.

TABLE 6
Ranges for Formulation Compositions

| | Total | Composition | |
|---|---|---|---|
| Ingredient | Dose (g) | (solids %) | Function |
| Na₂SO₄ | 28.02-42.02 | 56.58-84.86 | Active |
| MgSO₄ | 2.56-3.84 | 5.16-7.76 | Active |
| K₂SO₄ | 5.01-7.51 | 10.11-15.17 | Active |
| Sodium Benzoate | 0.11-0.16 | 0.22-0.32 | Preservative |
| Flavoring agents | 2.65-2.75 | 4.36-6.54 | Flavoring |
| Sucralose, NF | 2.16-2.24 | 3.55-5.33 | Flavoring |

### EQUIVALENTS

The foregoing description is illustrative of the certain embodiments shown. It is not intended to limit the present invention to the specific formulations shown and described, but instead it will be appreciated that adaptations and modifications will become apparent from the present disclosure and are intended to be within the scope of the claims. For example, small amounts of sodium chloride, potassium chloride and or bicarbonate may be added to consider patient needs.

1. A composition for inducing purgation of the colon of a patient the composition comprising from 0.01% to 0.1% of a sweetener and the composition for inducing purgation of the colon of a patient having a perceived saltiness equivalent to from 0.2% to 2.6% sodium chloride in water, wherein the composition excludes digestible sweeteners, and wherein the composition excludes phosphate.

2. The composition of claim 1 wherein the sweetener is selected from the group consisting of a chlorinated sucrose isomer, acesulfame potassium, saccharin, and mixtures thereof.

3. The composition of claim 1, where the chlorinated sucrose isomer is Sucralose.

4. The composition of claim 1, where the sweetener is a mixture of chlorinated sucrose isomer and acesulfame potassium in a 1 to from 4 to 6 ratio by weight, respectively.

5. The composition of claim 1, comprising from 0.04% to 0.09% of the sweetener and where the perceived saltiness of the composition for inducing purgation is equivalent to from 1.8% to 2.2% sodium chloride in water.

6. The composition of claim 1, comprising from 0.04% to 0.06% of the sweetener.

7. The composition of claim 1, comprising from 0.01% to 0.04% of the sweetener and where the perceived saltiness of the bowel cleanser is equivalent to from 0.2% to 0.6% sodium chloride in water.

8. The composition of claim 7, comprising from 0.01% to 0.03% of the sweetener.

9. The composition of claim 1, further comprising a flavoring selected from the group consisting of mangosteen, cola, ginger ale, and combinations thereof.

10. The composition of claim 9, including from 0.3% to 2.3% of the flavoring.

11. The composition of claim 1, where the composition for inducing purgation of the colon of a patient further comprises a polyethylene glycol.

12. A method for reducing saltiness of an orally consumed substance having a perceived saltiness equivalent to from 0.2 to 2.6% sodium chloride in water, comprising:combining the orally consumed substance with from 0.01% to 0.1% of a sweetener selected from the group consisting of a chlorinated sucrose isomer, acesulfame potassium, saccharin, and mixtures thereof.

13. The method of claim 12, where the orally consumed substance comprises a composition for inducing purgation of the colon of a patient.

14. The method of claim 13, where the composition for inducing purgation of the colon of a patient comprises polyethylene glycol.

15. A method for improving the palatability of a composition for inducing purgation of the colon of a patient, comprising:combining a concentration of a composition for inducing purgation of the colon of a patient having a perceived saltiness equivalent to from 0.2% to 2.6% sodium chloride in water of the composition for inducing purgation of the colon of a patient with from about 0.01 to about 0.07% of a sweetener selected from the group consisting of a chlorinated sucrose isomer, acesulfame potassium, saccharin, and mixtures thereof.
